(19) United States
(12) Patent Application Publication
(10) Pub. No.: US 2003/0216272 A1
(43) Pub. Date: Nov. 20, 2003

(54) Premoistened wipe comprising a detergent composition with a soil entrainment system

(75) Inventors: Alan Edward Sherry, Cincinnati, OH (US); Mary Vjayarani Barnabas, West Chester, OH (US); Robert Allen Godfroid, West Chester, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/349,733

(22) Filed: Jan. 23, 2003

Related U.S. Application Data
(63) Continuation-in-part of application No. 09/655,221, filed on Sep. 5, 2000.
(63) Continuation-in-part of application No. 09/671,718, filed on Sep. 27, 2000.
(63) Continuation-in-part of application No. 09/671,080, filed on Sep. 27, 2000, now Pat. No. 6,559,116.
Application No. 09/671,080 is a continuation-in-part of application No. 09/655,221, filed on Sep. 5, 2000.
(60) Provisional application No. 60/156,289, filed on Sep. 27, 1999.
(60) Provisional application No. 60/156,286, filed on Sep. 27, 1999.
(60) Provisional application No. 60/156,289, filed on Sep. 27, 1999.

(51) Int. Cl. C11D 17/00

(52) U.S. Cl. 510/295; 510/438; 510/499

(57) ABSTRACT

The present invention provides a pre-moistened cleaning wipe which are impregnated with a cleaning solution. The cleaning solution generally comprises a soil entrainment system which comprises one or more modified polyamine compounds, one or more modified polyacrylamide compounds and such that the pH of the solution is between 5 and 11. The pre-moistened cleaning wipe effectively cleans hard surfaces without unacceptable filming and/or streaking residue on the surface

The present invention also provides a hard surface cleaning compositions that effectively clean and reduce microorganisms on a surface without resulting in unacceptable filming and/or streaking residue on the surface, generally comprises (a) an effective amount of soil entrainment system which comprises one or more modified polyamine compounds, one or more modified polyacrylamide compounds, (b) an antimicrobial agent which is a polymeric biguanide and such that the pH of the solution is between 5 and 11.

### CROSS REFERENCE TO RELATED APPLICATIONS

This application is a continuation-in-part of U.S. application Ser. No. 09/655,221 filed on Sep. 5, 2000, which claims the benefit of U.S. Provisional Application No. 60/156,289 filed on Sep. 27, 1999. This application is also a continuation-in-part of U.S. application Ser. No. 09/671,718 filed on Sep. 27, 2000, which claims the benefit of U.S. Provisional Application No. 60/156,286 filed on Sep. 27, 1999. This application is also a continuation-in-part of U.S. application Ser. No. 09/671,080 filed on Sep. 27, 2000, which is a continuation-in-part of U.S. application Ser. No. 09/655,221 filed on Sep. 5, 2000, which claims the benefit of U.S. Provisional Application No. 60/156,289 filed on Sep. 27, 1999.

### TECHNICAL FIELD

This application relates to premoistened wipe compositions and articles comprising a detergent composition containing a soil entrainment system for cleaning hard surfaces. This application also relates to a process of using such premoistened wipe.

### BACKGROUND OF THE INVENTION

Premoistened wipes have become a consumer-accepted means for cleaning hard surfaces such as kitchen counters, bathroom sinks and floors throughout the home. Premoistened wipes offer improved usage convenience and better hygiene relative to traditional cleaning systems. However, they can have some negatives relative to systems that first dose liquid and then use a cleaning implement such as a sponge or disposable pad for wiping and removing soil.

One of the issues associated with premoistened wipes is that as they become increasingly dirty, they tend to redeposit much of the picked-up soil onto the surface to be cleaned. This, in turn, reduces the capacity of the premoistened wipe for additional soil removal. Good cleaning and soil retention properties are of paramount importance since premoistened wipes have limited fluid capacity and are typically used without the benefit of a rinse step. Poorer cleaning and/or excessive soil redeposition means that multiple wipes are often necessary to complete a cleaning task, and this significantly reduces their real and/or perceived consumer value. Particulate soils, such as clay, sand, dirt, lint, and humic soils are ubiquitous in consumer homes and can have a variety of origins. The difficulty with particulate soils is not their dissolution, but rather their removal from the surface. In no rinse situations, the particulate soils are suspended, but reappear upon drying as streaking and hazing on the “cleaned surface”. Wiping the surface with a premoistened wipe can remove some of the particulate soil but often also redistributes the particulate soil over the surface being cleaned. Floors, glass (windows, mirrors and the like) and wood (polished or unpolished) surfaces are particularly susceptible to particulate matter and particulate matter redeposition.

Consequently, there remains the need for a premoistened wipe that can readily and conveniently remove particulate soil from a hard surface and prevent soil redeposition, without causing filming and streaking issues and without any rinsing steps.

### SUMMARY OF THE INVENTION

The present invention meets the aforementioned needs in that it has been discovered that a soil entrainment system included as part of the detergent solution in the premoistened wipe traps the particulate soil, facilitating its removal from the surface, thereby dramatically improving the overall cleaning ability of the premoistened wipe. It has also surprisingly been found that the benefits of the soil entrainment system are greater when the soil entrainment system is directly incorporated onto a cleaning substrate than when it is sprayed onto the surface to be cleaned and wiped and removed using cleaning implements such as sponges or absorbent disposable pads. The soil entrainment system reduces the cleaning deficiency provided by the premoistened wipe thus improving its consumer appeal. Finally, it has been found that premoistened wipes and liquid compositions that additionally comprise polymeric biguanides provide an even better end result and performance along with strong antimicrobial properties. The present invention relates a premoistened wipe comprising:

a) at least one layer of a nonwoven substrate wherein said nonwoven substrate is impregnated with a cleaning composition loaded at weight factor of from about 1:1 to about 15:1, said cleaning composition comprising an effective amount of a soil entrainment system and wherein said soil entrainment system is selected from the group consisting of

1) one or more modified polyamine compounds, said modified polyamine compounds are selected from:

i) (PA)_{w}(T)ₓ;

ii) (PA)_{w}(L)_{z};

iii) [(PA)_{w}(T)ₓ]_{y}[L]_{z}; and

iv) mixtures thereof;

wherein PA is a grafted or non-grafted, modified or unmodified polyamine backbone unit, T is an amide-forming polycarboxylic acid crosslinking unit, and L is a non-amide forming crosslinking unit; provided that for compounds of type (i) and (iii) the indices w and x have values such that the ratio of w to x is from 0.8:1 to 1.5:1; for compounds of type (ii) the indices w and z have values such that said modified polyamine compound comprises from about 0.05 to about 2 parts by weight of said L unit; for compounds of type (iii) the indices y and z have values such that said modified polyamine compound comprises from about 0.05 to about 2 parts by weight of said L unit; and

2) one or more modified polyacrylamide compounds of the formula:

—[CR₂—CR₂]ₙ—

wherein each R unit is independently selected from the group consisting of hydrogen, C₁-C₁₂ alkyl, C₆-C₁₂ aryl, CON(R′)₂, and D; wherein each D unit is independently selected from the group consisting of CO₂N(R′)ₘ CON(R′)CH₂CON(R′)₂, OCON(R′)₂, and CO₂(CH₂)_{q}N(R′)ₘ, wherein each R′ is selected from the group consisting of hydrogen, C₁-C₁₂ alkyl, or both R′ units can be taken together to form a ring comprising 4-6 carbon atoms; q is an integer from 0 to 5; m is either 2 or 3 and n is a numbers selected such that said modified polyacrylamide compounds have an average molecular weight of from about 20,000 Daltons to about 10,000,000 Daltons; and

3) mixtures thereof; and

b) the balance adjunct ingredients;

wherein said cleaning composition has a pH as expressed from said premoistened wipe of from about 5 to about 11.

The advantages of the soil entrainment system in premoistened wipes will become apparent to those of ordinary skill in the art from a reading of the following detailed description, examples and the appended claims.

All documents cited herein are, in relevant part, incorporated herein by reference; the citation of any document is not to be construed as an admission that it is prior art with respect to the present invention.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All temperatures are in degrees Celsius (° C.) unless otherwise specified. All parts, ratios, and percentages herein, in the Specification, Examples, and Claims, are by weight and all numerical limits are used with the normal degree of accuracy afforded by the art, unless otherwise specified.

### DEFINITIONS

By ‘absorbent’ it is meant any material or laminate that can absorb at least about 1 gram of de-ionized water per gram of said material.

By ‘absorbent disposable cleaning pad’ it is meant an absorbent pad that is typically used for a cleaning job and then disposed of. Absorbent disposable cleaning pads can range from simple dry absorbent non-woven structures to multi-layered absorbent composites. While it is understood that some pad designs can be used, stored and re-used, the amount of re-use is limited and is typically determined by the ability of the pad to continue to absorb more liquid and/or dirt. Unlike conventional systems such as sponge mops, strip and string mops, which are considered fully re-usable, once saturated, an absorbent disposable pad is not designed to be reversed by the consumer to get it back to its original state.

By ‘implement’ or ‘cleaning implement’, it is meant any material used to contact a hard surface and remove soil form said surface. Cleaning implements include sponges, cloths, cellulose strings or strips, paper or commercially available paper towel, monolayer premoistened wipes, premoistened wipe laminates, and absorbent disposable cleaning pads. In several instances, cleaning implements are used in conjunction devices such as mop heads long pole attachments to improve the ease of cleaning process.

By ‘substrate’ or ‘non woven substrate’ it is meant any woven or non-woven material formed as a single structure during the manufacturing process, or present in the form of two or more material laminates. Within the context of the present invention, the term ‘substrate’ refers to the dry weight of the nonwoven structure.

By ‘pre-moistened wipe’ it is meant herein a substrate and an aqueous composition as described herein applied to the substrate.

By ‘synthetic material’ or ‘synthetic fibers’, it is meant herein a hydrophobic material based on synthetic organic polymers.

By ‘load factor’ it is meant the weight ratio of liquid detergent to dry substrate. For example, a premoistened wipe weighing 16 grams that comprises 14 grams of liquid detergent and 2 grams of (dry) substrate is said to have a load factor of 7:1.

By ‘binder’ or ‘latex’, it is meant any additive or treatment intended to provide strength, integrity, cohesion, or adhesion of fibers in a web and in process. The term includes fiber finishes that can be removed by soaking the web in an aqueous composition comprising either glycol ether solvents and/or C2-C4 alcohols.

### DETAILED DESCRIPTION OF THE INVENTION

The present invention relates to premoistened wipes comprising a layer of nonwoven substrate impregnated with a hard surface cleaning compositions comprising from about 0.001% to about 1.5%, preferably from about 0.005% to about 0.5%, more preferably from about 0.01% to about 0.1% by weight, of a soil entrainment system. The hard surface cleaning composition is in the form of an aqueous liquids or gels embedded in a nonwoven substrate. The composition may be an isotropic clear liquid, microemulsion, oil in water emulsion, or be in the form of lamellar or hexagonal phase or aqueous composition comprising vesicles.

Nonwoven Substrate:

The substrate herein can be a single layer of treated fibers, or can be a laminate comprising multiple layers of treated fibers produced together using processing techniques known in the art. Nonwoven substrates can be formed by dry forming techniques such as air-laying or wet laying such as on a papermaking machine. Other nonwoven manufacturing techniques such as hydroentangling, melt blown, spun bonded, needle punched and methods may also be used. So as to limit streaking and filming issues, the substrate is preferably substantially free of binder or latex.

The substrate can be made of synthetic materials, non-synthetic materials, and mixtures thereof. Synthetic materials, as used herein, include all polymers derived from polyethylene, polypropylene, polyester polymers and mixtures thereof. The synthetic materials may also include some level of polymer derived from tetrafluoroethylene.

The distribution of synthetic and non-synthetic fibers within the substrate web can be homogeneous or non-homogeneous. When the distribution of fibers is non-homogeneous, or when the substrate is a laminate (i.e., includes at least two layers of homogeneous or non-homogeneous fibers combined together to form the substrate), it is preferred that the exposed (top and bottom) surface areas of the wipes comprise a higher amount of synthetic fiber than is present in the overall substrate composition. Such a structure keeps a reservoir of fluid within the more absorbent non-synthetic structure, and sandwiched between the two areas of the wipe that are more hydrophobic; this results in more controlled release of the aqueous composition and better overall mileage for the wipe. Alternatively, the distribution of fibers can advantageously be made so that only one face of the substrate has more hydrophobic fibers than that of the overall composition. In this case, the substrate would be sided, providing one surface with increased synthetic content, and at least one surface made of cellulose or treated cellulose derivatives. Multi-laminate substrates that are particularly useful within the context of the present invention are described in greater details in U.S. patent application Ser. No. 09/671,718 to Sherry et al., filed Sep. 27, 2000, and assigned to The Procter & Gamble Company. The presence of hydrophobic material at the outer surface(s) of the premoistened wipe results in improved grease cleaning removal and retention during the cleaning process. It also is shown to improve the lubricity or glide of the substrate as it is wiped across a variety of hard surfaces. This can provide reassurance of “easy cleaning” in the context of a consumer goods product. The specific benefits associated with the incorporation of at least 20% synthetic fibers are more fully disclosed in U.S. patent application Ser. No. 10/267,266 to Barnabas et al., filed Oct. 9, 2002, and assigned to The Procter & Gamble Company. In a preferred embodiment, the combination of a premoistened wipe that comprises a substrate with synthetic and non-synthetic fibers along with a cleaning solution that comprises the soil entrainment system is found to provide the best combination of grease and particulate cleaning. The benefit of the inclusion of non-synthetic fibers lies in the increased adsorption affinity of the soil entrainment system to the substrate.

Suitable non-synthetic materials are man-made fibers and natural fibers. The term man-made fiber, as used herein, denotes fibers manufactured from cellulose, either derivative or regenerated. They are distinguished from synthetic fibers, which are based on synthetic organic polymers. A derivative fiber, as used herein, is one formed when a chemical derivative of a natural polymer, e.g., cellulose, is prepared, dissolved, and extruded as a continuous filament, and the chemical nature of the derivative is retained after the fiber formation process. A regenerated fiber, as used herein, is one formed when a natural polymer, or its chemical derivative, is dissolved and extruded as a continuous filament, and the chemical nature of the natural polymer is either retained or regenerated after the fiber formation process. Typical examples of man made fibers include: regenerated viscose rayon and cellulose acetate. Preferred man-made fibers have a fineness of about 0.5 dtex to about 3.0 dtex, more preferably of about 1.0 dtex to about 2.0 dtex, most preferably of about 1.6 dtex to about 1.8 dtex.

Suitable, natural fibers are selected from the group consisting of wood pulp, cotton, hemp, and the like. Man-made fibers are preferred herein due to their high consumer acceptance and their cheap and typically ecological production. Importantly, man-made fibers and in particular cellulose derived man-made fibers exhibit a high biodegradability, hence are environment friendly after disposal. Natural fibers can be preferred because they do not require the modifications needed to create the man-made fibers. As such natural fibers can provide cost advantages.

In a preferred embodiment, the man-made fiber for use in the substrate herein is a hydrophilic material, such as TENCEL® rayon, LENZING AG® rayon, micro-denier rayon, and LYOCELL®. Hydrophilic man-made fiber material, when at least partially present in the substrate herein, has been found to allow for increased load factor (described hereinafter) of the aqueous chemical composition applied to the substrate. Indeed, it has been found that a man-made fiber-containing substrate can incorporate more aqueous cleaning composition than a purely synthetic substrate. Furthermore, it has been found that a pre-moistened wipe comprising man-made fiber shows a slower release of the composition impregnated thereon during use as compared to a purely synthetic substrate. By slowing the release of the composition, the area that can be treated with the pre-moistened wipe is significantly increased. Additionally, the slower release ensures improved even-ness of solution distribution and better coverage of the surfaces treated.

Suitable, man-made fibers are commercially available under the trade name LYOCELL® fibers that are produced by dissolving cellulose fibers in N-methylmorpholine-N-oxide and which are supplied by Tencel Fibers United Kingdom.

Preferred man made fibers used for the present invention are selected from the group consisting of viscose rayon, high absorbency rayon, TENCEL® rayon, LENZING AG® rayon and mixtures thereof. For substrate laminates, treated pulp provides a low cost and convenient reservoir of fluid. It is understood that the specific choice of cellulose and cellulosic derivatives will depend on the desired cleaning and absorbency characteristics and associated costs.

The substrate preferably has a weight of from about 20 gm⁻² to about 300 gm⁻², preferably of at least about 20 gm⁻² and less than about 150 gm⁻², more preferably between about 20 gm⁻² and about 120 gm⁻², and most preferably from about 30 gm⁻² to about 120 gm⁻². The substrate may have any suitable caliper. Typically, when the substrate is made via an hydroentanglement process, the average substrate caliper is less than about 1.2 mm, preferably from about 0.1 mm to about 1.0 mm at a pressure of about 0.1 pounds per square inch (about 0.007 kilograms per square meter). The substrate caliper is measured according to standard EDANA nonwoven industry methodology, reference method #30.4-89.

In addition to the fibers used to make the substrate, the substrate can comprise other components or materials added thereto as known in the art, such as finishing agents and opacifying agents. For example titanium dioxide could be used to improve the optical characteristics of the substrate.

As previously discussed, the substrate is impregnated with a cleaning composition comprising a soil entrainment system and detergent adjuncts at a load factor of from about 1:1 to about 15:1, preferably from about 1.2:1 to about 12:1, more preferably from about 1.4:1 to about 10:1.

Cleaning Compositions:

The premoistened wipes of the present invention further comprise a cleaning composition. The cleaning composition can preferably comprise ingredients selected from the group consisting of surfactants, buffers, solvent, perfume, suds suppressor, antimicrobial agents, and mixtures thereof.

The cleaning composition ingredients will be present in varying amounts depending either upon their function in the cleaning composition or desired concentration in solution or on the surface to be cleaned. Furthermore, it is preferred to use cleaning ingredients that are compatible with the soil entrainment system and cause insignificant, preferably no streaking or hazing on the cleaned surface.

Surfactant

Typically, the cleaning composition will include one or more surfactant. The surfactant can be chosen from those that are typically used in hard surface cleaning. Preferably, at least one surfactant is selected from the group consisting of nonionic, zwitterionic, amphoteric and mixtures thereof. Anionic surfactants can be used but are less preferable as they tend to bind or interact with the soil entrainment system of the invention. Examples of suitable surfactants are described in McCutcheon's Vol. 1: Emulsifiers and Detergents, North American Ed., McCutheon Division, MC Publishing Co., 1999, which is incorporated herein by reference.

Suitable anionic surfactants typically comprise a hydrophobic chain containing from about 8 to about 18 carbon atoms, preferably from about 8 to about 16 carbon atoms, and typically include a sulfonate or carboxylate hydrophilic head group.

Suitable anionic surfactants include the C₈- C₁₈ alkyl sulfonates, C₁₀-C₁₄ linear or branched alkyl benzene sulfonates, C₁₀₋₁₄ alkyl sulfates and ethoxysulfates (e.g., STEPANOL AM® from Stepan), C₉-C₁₅ alkyl ethoxy carboxylates (NEODOX® surfactants available from Shell Chemical Corporation). Suitable commercially available sulfonates are available from Stepan under the tradename BIO-TERGE PAS-8® as well as from the Witco Corporation under the tradename WITCONATE NAS-8®, and HOSTAPUR SAS® from Hoechst, Aktiengesellschaft, D-6230 Frankfurt, Germany.

Suitable nonionic surfactants typically comprise a hydrophobic chain containing from about 8 carbon atoms to about 18, preferably from about 8 to about 16, carbon atoms, and typically include either a saccharide or alkoxylate end group.

One type of highly preferred nonionic surfactants are the alkylpolysaccharides that are disclosed in U.S. Pat. No. 5,776,872 issued Jul. 7, 1998, to Giret et al; in U.S. Pat. No. 5,883,059, issued Mar. 16, 1999, to Furman et al.; in U.S. Pat. No. 5,883,062 issued Mar. 16, 1999, to Addison et al., and U.S. Pat. No. 5,906,973, issued May 25, 1999, to Ouzounis et al.

Suitable alkylpolysaccharides are disclosed in U.S. Pat. No. 4,565,647, to Llenado, issued Jan. 21, 1986, which have a hydrophobic group containing from about 6 to about 30 carbon atoms, preferably from about 10 to about 16 carbon atoms as well as a polysaccharide, such as a polyglycoside, hydrophilic group. For acidic or alkaline cleaning compositions/solutions suitable for use in no-rinse methods, the preferred alkyl polysaccharide preferably comprises a broad distribution of chain lengths, as these provide the best combination of wetting, cleaning, and low residue upon drying. This “broad distribution” is defined by at least about 50% of the chainlength mixture comprising from about 10 carbon atoms to about 16 carbon atoms. In a preferred embodiment, the alkyl group of the alkyl polysaccharide consists of a mixtures of chainlength, comprising from about 6 to about 18 carbon atoms, preferably from about 8 to about 16 carbon atoms, and a hydrophilic group containing from about one to about 1.5 saccharide, preferably glucoside, groups per molecule. This “broad chainlength distribution” is defined by at least about 50% of the chainlength mixture comprising from about 10 carbon atoms to about 16 carbon atoms. A broad mixture of chain lengths, particularly C₈-C₁₆, is highly desirable relative to narrower range chain length mixtures, and particularly versus lower (i.e., C₈-C₁₀ or C₈-C₁₂) chainlength alkyl polyglucoside mixtures. It is also found that the preferred C₈₋₁₆ alkyl polyglucoside provides much improved perfume solubility versus lower and narrower chainlength alkyl polyglucosides, as well as other preferred surfactants, including the C₈-C₁₄ alkyl ethoxylates. Any reducing saccharide containing 5 or 6 carbon atoms can be used, e.g., glucose, galactose and galactosyl moieties can be substituted for the glucosyl moieties. (optionally the hydrophobic group is attached at the 2-, 3-, 4-, etc. positions thus giving a glucose or galactose as opposed to a glucoside or galactoside.) The intersaccharide bonds can be, e.g., between the one position of the additional saccharide units and the 2-, 3-, 4-, and/or 6- positions on the preceding saccharide units. The glycosyl is preferably derived from glucose.

In the alkyl polyglycosides, the alkyl moieties can be derived from the usual sources like fats, oils or chemically produced alcohols while their sugar moieties are created from hydrolyzed polysaccharides. Alkyl polyglycosides are the condensation product of fatty alcohol and sugars like glucose with the number of glucose units defining the relative hydrophilicity. As discussed above, the sugar units can additionally be alkoxylated either before or after reaction with the fatty alcohols. Such alkyl polyglycosides are described in detail in International Patent Application publication No.WO 86/05199 to Arshad Malik, published on Sep. 12, 1986. Technical alkyl polyglycosides are generally not molecularly uniform products, but represent mixtures of alkyl groups and mixtures of monosaccharides and different oligosaccharides. Alkyl polyglycosides (also sometimes referred to as “APG's”) are preferred for the purposes of the invention since they provide additional improvement in surface appearance relative to other surfactants. The glycoside moieties are preferably glucose moieties. The alkyl substituent is preferably a saturated or unsaturated alkyl moiety containing from about 8 to about 18 carbon atoms, preferably an average of from about 8 to about 11 carbon atoms or a mixture of such alkyl moieties. A few examples of highly preferred C10 alkyl polyglucoside are GLUCOPON®425, PLANTAREN 2000®, PLANTAREN 2000®, and PLANTAREN 2000 N UP®, available from Cognis Corporation (Postfach 101100, D 40191 Dusseldorf, Germany). C₈-C₁₆ alkyl polyglucosides are commercially available (e.g., SIMUSOL® surfactants from Seppic Corporation, 75 Quai d'Orsay, 75321 Paris, Cedex 7, France, and GLUCOPON®625 available from Cognis.

Another class of nonionic surfactants suitable for the present invention is alkyl ethoxylates. The alkyl ethoxylates of the present invention are either linear or branched, and contain from about 8 carbon atoms to about 14 carbon atoms, and from about 4 ethylene oxide units to about 25 ethylene oxide units. Examples of alkyl ethoxylates include NEODOL® 91-6, NEODOL 91-8® supplied by the Shell Corporation (P.O. Box 2463, 1 Shell Plaza, Houston, Tex.), and ALFONIC® 810-60 supplied by Vista corporation, (900 Threadneedle P.O. Box 19029, Houston, Tex.). More preferred surfactants are the alkyl ethoxylates comprising from about 9 to about 12 carbon atoms, and from about 4 to about 8 ethylene oxide units. These surfactants offer excellent cleaning benefits and work synergistically with the required hydrophilic polymers. A most preferred alkyl ethoxylate is C₁₁EO₅, available from the Shell Chemical Company under the trademark NEODOL® 1-5.

Alternative nonionic detergent surfactants for use herein are alkoxylated alcohols generally comprising from about 6 to about 16 carbon atoms in the hydrophobic alkyl chain of the alcohol. Typical alkoxylation groups are propoxy groups or propoxy groups in combination with ethoxy groups. Such compounds are commercially available under the tradename ANTAROX® available from Rhodia (CN 7500, Cranberry, N.J.). with a wide variety of chain length and alkoxylation degrees. Block copolymers of ethylene oxide and propylene oxide can also be used and are available from BASF under the tradename PLURONIC®. Preferred nonionic detergent surfactants for use herein are according to the formula R(X)ₙH, were R is an alkyl chain having from about 6 to about 16 carbon atoms, preferably from about 8 to about 12, X is a propoxy, or a mixture of ethoxy and propoxy groups, n is an integer of from about 4 to about 30, preferably from about 5 to about 8. Other non-ionic surfactants that can be used include those derived from natural sources such as sugars and include C₈-C₁₆ N-alkyl glucose amide surfactants.

Also so suitable for use in the present invention are the fluorinated nonionic surfactants. One particularly suitable fluorinated nonionic surfactant is Fluorad F170 (3M). Fluorad F170 has the formula:

₈F₁₇.SO₂N(C₂H₅)(CH₂CH₂O)

Also suitable for use in the present invention are silicone-containing surfactants. One example of these types of surfactants is Silwet L7604 avalaible from Union Carbide.

Some preferred commercially available surfactants include Neodol 11-5, Nonidet SF-3, Nonidet SF-5, (all Shell Chemical), C8 sulfonate (Witconate NAS-8) C11-18 APG (Henkel), Fluorad F170 (3M).

In general, the level of optional surfactants, when present in the compositions herein is from about 0% to about 0.25%, preferably from about 0.001% to about 0.2%, more preferably from about 0.01% to about 0.15%, by weight of the composition.

Buffer

The buffering agent may be an active detergent in its own right, or it may be a low molecular weight, organic or inorganic material that is used in this composition solely for maintaining the desired pH. The buffer can be alkaline, acidic or neutral. Preferred buffering agents for compositions of this invention are nitrogen-containing materials. Some examples are amino acids such as lysine or lower alcohol amines like mono-, di-, and tri-ethanolamine. Other preferred nitrogen-containing buffering agents are Tri(hydroxymethyl)amino methane (HOCH2)3CNH3 (TRIS), 2-amino-2-ethyl-1,3-propanediol, 2-amino-2-methyl-propanol, 2-amino-2-methyl- 1,3-propanol, disodium glutamate, N-methyl diethanolamide, 2-dimethylamino-2-methylpropanol (DMAMP), 1,3-bis(methylamine)-cyclohexane, 1,3-diamino-propanol N,N′-tetra-methyl-1,3-diamino-2-propanol, N,N-bis(2-hydroxyethyl)glycine (bicine) and N-tris (hydroxymethyl)methyl glycine (tricine). Other suitable buffers include ammonium carbamate, citric acid, acetic acid. Mixtures of any of the above are also acceptable. Useful inorganic buffers/alkalinity sources include ammonia, the alkali metal carbonates and alkali metal phosphates, e.g., sodium carbonate, sodium polyphosphate. For additional buffers see McCutcheon's EMULSIFIERS AND DETERGENTS, North American Edition, 1999, McCutcheon Division, MC Publishing Company Kirk and WO 95/07971 both of which are incorporated herein by reference.

Preferred buffers include, but are not limited to, ammonia, methanol amine, ethanol amine, 2-amino-2-methyl- 1-propanol, 2-dimethylamino-2-methyl- 1 -propanol, 1,3-bis(methylamine)-cyclohexane, acetic acid, glycolic acid and the like. Most preferred among these are ammonia, 1,3-bis(methylamine)-cyclohexane, 2-dimethylamino-2-methyl-1-propanol and acetic acid.

In one preferred aspect the composition of the present invention in order to minimize streaking/filming problems, the buffering is provided, at least in part, by volatile materials whose molecular weight is less than about 400 g/mole.

The composition will preferably contain about 0.001%, even more preferably still, at least about 0.005% by weight of the composition of buffering agent. The composition will also preferably contain no more than about 1%, more preferably no more than about 0.75%, even more preferably, no more than about 0.5% by weight of the composition of buffering agent.

Aqueous Liquid Carrier—The compositions of the present invention contain an aqueous liquid carrier, preferably from about 10% to about 99.9%, preferably from about 30% to about 98%, by weight the composition.

It is preferred that any water in the composition, such as in premixed or ready to use solutions, is deionized or softened water. However, conventional tap water can be used but is not preferred.

Solvent

The compositions, optionally, can also contain one, or more, solvents at effective levels, typically, when present, no less than about 0.25%, and, at least about, in increasing order of preference, about 0.5% and about 3.0%, and no more than about, in increasing order of preference, about 7% and about 5% by weight of the composition.

Such solvents typically have a terminal C₃-C₆ hydrocarbon attached to from one to three ethylene glycol or propylene glycol moieties to provide the appropriate degree of hydrophobicity and, preferably, surface activity. Examples of commercially available hydrophobic cleaning solvents based on ethylene glycol chemistry include mono-ethylene glycol n-hexyl ether (HEXYL CELLOSOLVE® available from Dow Chemical (1691 N. Swede Road, Midland, Mich.). Examples of commercially available hydrophobic cleaning solvents based on propylene glycol chemistry include the di-, and tri-propylene glycol derivatives of n-propyl and n-butyl alcohol, which are available from Arco Chemical (3801 West Chester Pike, Newtown Square, Pa. 19073) and Dow Chemical under the trade names ARCOSOLV® and DOWANOL® respectively.

In the context of the present invention, preferred solvents are selected from the group consisting of propylene glycol n-propyl ether, di-propylene glycol n-propyl ether, propylene glycol n-butyl ether, di-propylene glycol n-propyl ether , di-propylene glycol n-butyl ether; tri-propylene glycol n-butyl ether; ethylene glycol n-butyl ether; di-ethylene glycol n-butyl ether, ethylene glycol n-hexyl ether and di-ethylene glycol n-hexyl ether, methanol, ethanol, isopropanol, n-butanol, iso-butanol, pentanol, 2-methyl-1-butanol, 2-butanone, methoxymethanol, methoxyethanol, methoxy propanol, ethoxypropanol, propoxypropanol, ethoxybutanol and mixtures thereof. “Butyl” includes normal butyl, isobutyl and tertiary butyl groups. More prefered solvents include ethanol, is-propanol, propylene glycol n-propyl ether and propylene glycol n-butyl ether and ethylene glycol n-hexyl ether. In compositions intended to provide antimicrobial benefits, ethylene glycol n-hexyl ether is highly desirable.

The amount of solvent can vary depending on the amount of other ingredients present in the composition. The solvent is normally helpful in providing good cleaning.

Antimicrobial Agents

The compositions, optionally, can also contain one or more, antimicrobial agents. The antimicrobial agents are selected from those typically used in hard surface cleaning with the proviso that they are compatible with the soil entrainment system and cause insignificant, preferably no streaking or hazing on the cleaned surface upon drying. Suitable antimicrobial agents include quaternary amines, such as didecyldimethylammonium chloride (Bardac 2250, Lonza); chlorhexidine digluconate, methylbenzethonium chloride, dodecyltriphenyphosphonium bromide and mixtures thereof.

In a preferred embodiment, the disinfectant is selected from the group consisting of polymeric biguanides. Biguanide agents are characterized in comprising at least one, preferably about 2 or more, biguanide moieties according to the following formula:

—NH—C(═NH)—NH—C(═NH)—NH—

The polymeric biguanides are oligo- or poly (alkylene biguanides) or salts thereof or mixtures thereof. More preferred biguanides are oligo- or poly (hexamethylene biguanides) or salts thereof or mixtures thereof. In a still more preferred embodiment said polymeric biguanide is a poly (hexamethylene biguanide) or salt thereof according to the following formula:

—[—(CH₂)₃—NH—C(═NH)—NH—C(═NH)—NH—(CH₂)₃—]ₙ—

wherein n is an integer selected from about 1 to about 50, preferably about 1 to about 20, more preferably about 9 to about 18. More preferably said biguanide is a salt of a poly (hexamethylene biguanide) according to the following formula:

—[—(CH₂)₃—NH—C(═NH)—NH—C(═NH)—NH—(CH₂)₃—]ₙ—.nHX

wherein n is an integer selected from about 1 to about 50, preferably about 1 to about 20, more preferably about 9 to about 18 , and HX is salt component, preferably HCl.

The polymeric biguanides function synergistically with the soil entrainment system of the present invention. U.S. Patent Application serial No. 60/328,008 to Barnabas et al., filed Oct. 9, 2001, and assigned to The Procter & Gamble Company, has previously recognized that filming and streaking issues are observed when the pH of premoistened wipe cleaning compositions comprising polymeric biguanides is higher than about pH 7. Use of the soil entrainment system, allows for incorporation of polymeric biguanides at neutral and alkaline pH without causing filming and streaking issues. More specifically, the polymeric biguanides actually enhance the cleaning performance of compositions comprising the soil entrainment system. Thus, while most antimicrobial agents leave visible residue, it is found that, in the presence of the soil entrainment system, compositions comprising the polymeric biguanides disinfect, clean better and reduce observable filming and streaking. Surface appearance benefits are also noted for aqueous cleaning compositions that combination of soil entrainment system and polymeric biguanide. The benefits can be achieved by spraying or otherwise dispensing the compositions on the surface to be cleaned, followed by wiping with either a sponge, paper towel, string mop, strip mop, or with an absorbent disposable pads such as those sold under the tradename SWIFFER WETJET®, Clorox READY MOP® or GRAB IT GO MOP®. In another embodiment, the composition is made as a concentrate and then diluted as needed. In a preferred embodiment, the concentrate is formulated with soil entrainment system and at least one polymeric biguanide, such that upon dilution to form a 1× product, significant antimicrobial benefits are retained. The consumer can therefore use the concentrated composition either neat for tough cleaning jobs, or dilute, for lighter cleaning, and still have antimicrobial reassurance for all tasks. The concentrate is preferably made such that after a dilution of from about 1:10 to about 1:100 with water, significant antimicrobial or sanitization benefits are retained. The preferred dilution levels suggest that the concentrate contain from about 1% to about 20% polymeric biguanide by weight of the composition. In one embodiment, compositions comprising polymeric biguanides, when present, have a pH less than about 10, preferably a pH from about 5 to about 9, more preferably from about pH 5.5 to about pH 8.5, more preferably from about pH 7 to about pH 8.5. It is also found that the presence of the soil entrainment agent can enhance the antimicrobial properties of premoistened wipes or other liquid compositions comprising polymeric biguanides or other antimicrobials described previously herein. Though not wishing to be limited by theory, the unexpected antimicrobial benefits associated with the inclusion of the entrainment system herein are believed to be due to the ability of the entrainment system to adhere both to the nonwoven substrate and to germs (bacteria, viruses or fungi) that must be removed from the cleaning surface. As such the entrainment system more effectively transfers microorganisms from the surface to be cleaned to the cleaning implement, thereby reducing the number of bacteria remaining on the surface against which the antimicrobial composition must act. In essence, it is believed that the soil entrainment system functions in the same way versus microorganisms as it does against lifeless particulate. Those skilled in the art will recognize that the levels of antimicrobial agent and polymeric biguanide will depend on the magnitude of the antimicrobial benefits sought.

Suds Suppressor—The composition of the present invention can optionally contain a suds suppressor. When present the suds suppressor is preferably present from about 0.0005% to about 0.01%, preferably from about 0.001% to about 0.005% by weight of the composition.

Suitable suds suppressors include, silicone suds suppressor such as silicone polymers and linear or branched C₁₀-C₁₈ fatty acids or alcohols, with silicone suds suppressor being preferred. One suitable suds supressor is Dow Corning silicone AF; other beneficial silicone suds suppressors include ANTIFOAM 1410® and silicone DB 110A®, also available from Dow Corning.

Another suitable suds suppressors is a mixture of polyethylene glycol stearate (4% Wt, CAS # 9004993); methylated silica (2% Wt, CAS # 67762907); Octamethyl cyclotetrasiloxane (2% Wt, CAS # 556672), available from Dow Corning.

Further examples of suitable suds suppressors can be found in co-pending U.S. Pat. No. 6,380,151 to Masters et al., issued Apr. 30, 2002 and assigned to The Procter & Gamble Company.

Soil Entrainment System

Suitable soil entrainment system of the present invention is described in U.S. patent application Ser. No. 09/671,080 to Godfroid et al., filed Sep. 27, 2000, and assigned to The Procter & Gamble Company. The entrainment system is comprised of one or more modified polyamine compounds, one or more modified polyacrylamide compounds and mixtures thereof.

1) Modified polyamine compounds—The soil entrainment system of the present invention may be comprised of one or more modified polyamines according to the present invention. The modified polyamines of the present invention which may comprise the soil entrainment system may be formulated as an admixture wherein a proportional amount of two or more compounds are combined to make up the soil entrainment system. Alternatively, the formulator may adjust the reaction conditions which form the modified polyamines of the present invention in order to create an admixture of suitable ingredients inter alia an admixture of polyamine fragments and/or partially crosslinked modified polyamines. Whether a formulated admixture or a product by process is used, or a mixture of both, the compounds which comprise the soil entrainment system of the present invention have the formula:

i) (PA)_{w}(T)ₓ;

ii) (PA)_{w}(L)_{z};

iii) [(PA)_{w}(T)ₓ]_{y}[L]_{z};

wherein PA is a grafted or non-grafted, modified or unmodified polyamine backbone unit, T is an amide-forming polycarboxylic acid crosslinking unit, and L is a non-amide forming crosslinking unit. For compounds of type (i) and (iii) the relative amounts of PA units and T units which are present are such that the molar ratio of PA units to T units is from 0.8:1 to 1.5:1. For compounds of type (ii) the relative amounts of PA units and L units which are present are such that the (PA)_{W}(L)_{z} comprises from about 0.05, preferably from about 0.3 to 2 parts by weight of said L units. Therefore, 1 part of a grafted or non-grafted, modified or unmodified polyamine backbone unit may be combined with from about 0.05, preferably from about 0.3 parts by weight of an L unit to about 2 parts by weight of an L unit to form a suitable modified polyamine compound. Likewise, for compounds of type (iii), crosslinked polyamines having the formula (PA)_{w}(T)ₓ may be combined with from about 0.05, preferably from about 0.3 parts by weight of an L unit to about 2 parts by weight of an L unit to form a suitable modified polyamine compound having the formula [(PA)_{w}(T)ₓ]_{y}[L]_{z}.

Polyamine Backbone (PA Units)

The modified polyamine compounds of the present invention comprise a Polyamine Backbone, PA unit, which can be optionally, but preferably grafted. The following are non-limiting examples of suitable PA units according to the present invention.

Polyalkyleneimine

A preferred PA unit according to the present invention are polyalkyleneimines and polyalkyleneamines having the general formula:

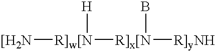

wherein R is C₂-C₁₂ linear alkylene, C₃-C₁₂ branched alkylene, and mixtures thereof preferably R is ethylene, 1,3-propylene, and 1,6-hexylene, more preferred is ethylene; B representing a continuation of the chain structure by branching. The indices w, x, and y are such that the molecular weight of said polyamines is from about 50,000 Daltons to about 15,000,000 Daltons, more preferably from about 350,000 Daltons to about 15,000,000 Daltons, even more preferably still from about 600,000 Daltons to about 15,000,000 Daltons. The index w typically has the value of y+1. PA units may be used as crude products or mixtures, and if desired by the formulator, these PA units may be used in the presence of small amounts of diamines as described herein above, wherein the amount of diamines, inter alia, ethylene diamine, hexamethylene diamine may be present up to about 10% by weight, of the PA unit mixture.

Co-polymeric Polyamines

Another example of a preferred PA unit according to the present invention are the polyvinyl amine homo-polymers or co-polymers having the formula:

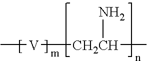

wherein V is a co-monomer, non-limiting examples of which include vinyl amides, vinyl pyrrolidone, vinyl imidazole, vinyl esters, vinyl alcohols, and mixtures thereof, all of which can be taken together or in combination with polyvinyl amine to form suitable co-polymerization products suitable for use in the soil entrainment system of the present invention.

The indices w, x, y, m (when present), and n, when present, are such that the molecular weight of said polyamines is from about 50,000 Daltons to about 15,000,000 Daltons, more preferably from about 350,000 Daltons to about 15,000,000 Daltons, even more preferably still from about 600,000 Daltons to about 15,000,000 Daltons.

Polyamine Backbone Modifications

Optionally, but preferably, the PA units of the present invention are modified either before or after reaction with a T unit or L unit crosslinking agent. The two preferred types of modifications are grafting and capping.

Preferably the PA units of the present invention are grafted, that is the PA unit is further reacted with a reagent which elongates said PA unit chain, preferably by reaction of the nitrogens of the PA backbone unit with one or more equivalents of aziridine (ethyleneimine), caprolactam, and mixtures thereof. Grafting units, in contrast to the “capping” units described herein below, can further react on themselves to provide PA unit chain propagation. An example of a preferred grafted PA unit of the present invention has the formula:

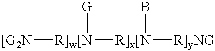

wherein R, B, w, x, and y are the same as defined herein above and G is hydrogen or an extension of the PA unit backbone by grafting. Non-limiting examples of preferred grafting agents are aziridine (ethyleneimine), caprolactam, and mixtures thereof. A preferred grafting agent is aziridine wherein the backbone is extended by units having the formula:

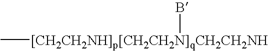

wherein B′ is a continuation by branching wherein the graft does not exceed about 12 units, preferably —CH₂CH₂NH₂ and the value of the indices p+q have the value from 0, preferably from about 1, more preferably from about 2 to about 7, preferably to about 5. Another preferred grafting unit is caprolactam.

The PA units of the present invention can be grafted prior to or after crosslinking with one or more T units described herein below, preferably the grafting is accomplished after crosslinking with said T unit. This allows the formulator to take advantage of the differential reactivity between the primary and secondary amino units of the PA unit backbone thereby allowing the formulator to controllably link said PA units and to also control the amount of subsequent branching which results from the grafting step.

Another optional but preferred PA unit modification is the presence of “capping” units. For example, a PA unit is reacted with an amount of a monocarboxylic acid, non-limiting examples of which are C₁-C₂₂ linear or branched alkyl, preferably C₁₀-C₁₈ linear alkyl inter alia lauric acid, myristic acid. The amount of capping unit which is reacted with the PA unit is an amount which is sufficient to achieve the desired properties of the formula. However, the amount of capping unit used is not sufficient to abate any further crosslinking or grafting which the formulator may choose to perform.

Crosslinking Units

Amide-forming T Crosslinking Units

T crosslinking units are preferably carbonyl comprising polyamido forming units. The T units are taken together with PA units to form crosslinked modified polyamine compounds having the formula (PA)_{w}(T)ₓ or [(PA)_{w}(T)ₓ]_{y}[L]_{z}.

A preferred embodiment of the present invention includes crosslinked PA units wherein a T unit provides crosslinking between two or more PA units to form a (PA)_{w}(T)ₓ polyamido crosslinked section. A preferred crosslinking T unit has the general formula:

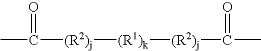

wherein R¹ is methylene, phenylene, and mixtures thereof, preferably methylene. The index k has the value from 2 to about 8, preferably to about 4. Preferred values of k are 2, 3, and 4. R² is —NH— thereby forming a urethane amide linkage when said R² comprising T units react with the backbone nitrogens of the PA units. The value of the index j is independently 0 or 1. The presence of R² units can result, for example, from the use of diisocyanates as crosslinking agents. Non-limiting examples of dibasic acids which are used as a source for T units in the above formula include succinic acid, maleic acid, adipic acid, glutaric acid, suberic acid, sebacic acid, and terephthalic acid. However, the formulator is not limited to crosslinking T units deriving from dibasic acids, for example, tribasic crosslinking T units, inter alia, citrate, may be used to link the PA units of the present invention.

Examples of (PA)_{w}(T)ₓ compounds according to the present invention are obtained by condensation of dicarboxylic acids inter alia succinic acid, maleic acid, adipic acid, terephthalic acid, with polyalkylene polyamines inter alia diethylenetriamine, triethylenetetramine, dipropylenetriamine, tripropylenetetramine wherein the ratio of the dicarboxylic acid to polyalkyleneamine is from 1:0.8 to 1:1.5 moles, preferably a ratio of from 1:0.9 to 1:1.2 moles wherein the resulting crosslinked material has a viscosity in a 50% by weight, aqueous solution of more than 100 centipoise at 25 ° C.

Non-amide Forming L Crosslinking Units

Another preferred embodiment of the polyamines of the present invention are (PA)_{w}(T)ₓ units which are further crosslinked by L units to form polyamido amines having the formula [(PA)_{w}(T)ₓ]_{y}[L]_{z}, or are reacted with PA units to form non-amide polyamines having the formula (PA)_{w}(L)_{z}.

The L units of the present invention are any unit which suitably crosslinks PA units or (PA)_{w}(T)ₓ units. Preferred L linking units comprise units which are derived from the use of epihalohydrins, preferably epichlorohydrin, as a crosslinking agent. The epihalohydrins can be used directly with the PA units or suitably combined with other crosslinking adjuncts non-limiting examples of which include alkyleneglycols, and polyalkylene polyglycols inter alia ethylene glycol, diethylene glycol, polyethylene glycol, propylene glycol, dipropylene glycol, polypropylene glycol, butylene glycol, hexanediol-1,6-glycerol, oligoglycerol, pentaerythrites, polyols which are obtained by the reduction of carbohydrates (sorbitol, mannitol), monosaccharides, disaccharides, oligosaccharides, polysaccharides, polyvinyl alcohols, and mixtures thereof.

For example, a suitable L unit is a dodecylene unit having the formula:

—(CH₂)₁₂—

wherein an equivalent of 1,12-dichlorododecane is reacted, for example, with a suitable amount of a PA unit to produce a polyamine which is crosslinked via dodecylene units. For the purposes of the present invention, L crosslinking units which comprise only carbon and hydrogen are considered to be “hydrocarbyl” L units. Preferred hydrocarbyl units are polyalkylene units have the formula:

—(CH₂)ₙ—

wherein n is from 1 to about 50.

Hydrocarbyl L units may be derived from hydrocarbons having two units which are capable of reacting with the nitrogen of the PA units. Non-limiting examples of precursors which result in the formation of hydrocarbyl L units include 1,6-dibromohexane, 1,8-ditosyloctane, and 1,14-dichlorotetradecane.

Further examples of preferred non-amide forming crosslinking L units are the units which derive from crosslinking units wherein epihalohydrin is used as the connecting unit. For example, 1,12-dihydroxydodecane is reacted with epichlorohydrin to form the bis-epoxide non-amide forming L unit precursor having the formula:

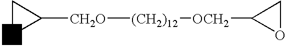

which when reacted with one or more PA units or (PA)_{w}(T)ₓ units results in an L crosslinking unit having the formula:

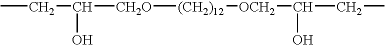

however, it is not necessary to pre-form and isolate the bis-epoxide, instead the crosslinking unit precursor may be formed in situ by reaction of 1,12-dihydroxydodecane 20 or other suitable precursor unit with epihalohydrin in the presence of grafted or ungrafted PA units or (PA)_{w}(T)ₓ units.

Other crosslinking L units which utilize one or more epihalohydrin connecting units include polyalkyleneoxy L units having the formula:

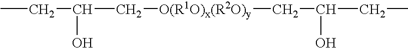

wherein R¹ is ethylene, R² is 1,2-propylene, x is from 0 to 100 and y is from 0 to 100. Another preferred unit which can comprise an L unit and which can be suitably combined with epihalohydrin connecting units include polyhydroxy units having the formula:

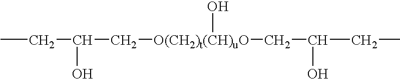

wherein the index t is from at least 2 to about 20 and the index u is from 1 to about 6. The formulator may also combine units to form hybrid L crosslinking units, for example, units having the formula:

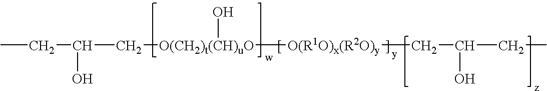

wherein the indices w and y are each independently from 1 to 50, z is units are present in a sufficient to suitably connect the polyhydroxy units and the polyalkyleneoxy units into the backbone without the formation of ether linkages.

The following is an example of an L linking group that comprises both a polyalkyleneoxy and a polyhydroxy unit.

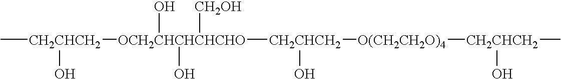

A further example of a preferred crosslinking L units are units which comprises at least two aziridine groups as connecting groups, for example an L unit having the formula:

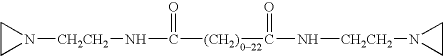

which can be used to link two (PA)_{w} units, two (PA)_{w}(T)ₓ units, or mixtures thereof.

The polyamines of the present invention may have varying final compositions, for example, (PA)_{w}(T)ₓ,[(PA)_{w}(T)ₓ]_{y}[L]_{z}, [(PA)]_{w}[L]_{z}, and mixtures thereof, wherein each PA unit may be grafted or ungrafted. The indices w and x have values such that the ratio of w to x is from 0.8: 1 to 1.5: 1; y and z have values such that said polyamido compound comprises from about 0.05, preferably to about 0.3 to 2 parts by weight of said L unit. In the cases wherein no crosslinking takes place the indices w and y will be equal to 1 and x and z will be equal to 0. In the case wherein no crosslinking occurs using L units, the index y is equal to 1 and z is equal to 0. In the case wherein no crosslinking occurs using T units, the indices w and y are equal to 1 and x is equal to 0.

A preferred embodiment of the present invention which comprises PA units, T units, and L units includes the reaction product of:

a) 1 part by weight, of a polyamine obtained by condensation of 1 mole of a dicarboxylic acid with a polyalkylene polyamine (i.e., diethylenetriamine) to the extent wherein at least about 10% of the —NH backbone hydrogens are unmodified by reaction with said dicarboxylic acid, then optionally reacting the obtained polyamine condensation product with up to 12 ethyleneimine units (i.e., grafting of the backbone using aziridine) per basic nitrogen atom; and

b) further reacting the product obtained in (a) with from 0.05, preferably from about 0.3 to about 2 parts by weight, of an L units, inter alia the reaction product of a polyalkylene oxide having from 8 to 100 alkylene oxide units with epichlorohydrin at a temperature of form about 20 ° C. to about 100 ° C.

The high molecular weight modified polyamine condensation products of the present invention (also referred to herein as “resins”) are preferably formed from the reaction of one or more grafted, cross-linked polyethyleneimines and one or more polyethylene and/or polypropylene glycol copolymers, wherein the resulting crosslinked modified polyamines (resins) have a final viscosity of more than or equal to 300 mPa-sec., preferably from 400 to 2,500 mPa-sec. when measured at 20° C. in a 20% aqueous solution. The modified polyamine compounds of the present invention are suitably described in U.S. Pat. No. 3,642,572 to Eadres et al., issued Feb. 15, 1972, U.S. Pat. No. 4,144,123 to Scharfet al., issued Mar. 13, 1979 and U.S. Pat. No. 4,371,674 to Hertel et al., issued Feb. 1, 1983. Examples of preferred modified polyamine are Lupasol SK and Lupasol SKA avalaible from BASF.

2 Modified Polyacrylaminde Polymers

The modified polyacrylamide polymers useful in the present invention have the formula:

—[CR₂—CR₂]ₙ—

wherein each R unit is independently hydrogen, C₁-C₁₂ alkyl, C₆-C₁₂ aryl, CON(R′)₂, and D as described herein below; preferably C₁-C₄ alkyl, hydrogen, D units and CON(R′)₂, more preferably hydrogen, D units and CON(R′)₂, wherein each R′ is independently selected from hydrogen, C₁-C₆ alkyl, or both R′units can be taken together to form a ring comprising 4-6 carbon atoms, preferably hydrogen, C₁-C₆ alkyl.

For the purposes of the present invention the term “homopolymeric” is defined as “a polymer backbone which is comprised of units having the same unit composition, i.e., formed from polymerization of the same monomer”. For the purposes of the present invention the term “copolymeric” is defined as “a polymer backbone which is comprised of units having a different unit composition, i.e., formed from the polymerization of two or more monomers”.

The number of D units present in the modified polyacrylamide polymers depends upon the formulation. For example, the number of D units will be adjusted to provide water solubility of the polymer. The molecular weight of the modified polyacrylamide polymers useful in the present invention are from about 20,000 Daltons to about 10,000,000 Daltons, more preferably from about 200,000 Daltons to about 15,000,000 Daltons, even more preferably still from about 350,000 Daltons to about 15,000,000 Daltons. Therefore the value of the index n is selected to provide the indicated molecular weight, and providing for a water solubility of least 100 ppm, preferably at least about 300 ppm, and more preferably at least about 1,000 ppm in water at ambient temperature which is defined herein as 25° C.

Each D is independently selected from the group consisting of, CO₂N(R′)ₘ, CON(R′)CH₂CON(R′)₂, OCON(R′)₂, CO₂(CH₂)_{q}N(R′)ₘ, preferably CO₂(CH₂)_{q}N(R′)ₘ, CO₂N(R′)ₘ; wherein R′ is as defined above, q is an integer from 0 to 5, preferably from 0 to 3, more preferably from 0 to 2, and m is either 2 or 3. One preferred D is the quaternary N substituted acrylamides, such as CO₂(CH₂)_{q}N⁺( R′)₃.

The indices m and n, when present, are such that the molecular weight of said polyamines is from about 20,000 Daltons to about 10,000,000 Daltons, more preferably from about 200,000 Daltons to about 15,000,000 Daltons, even more preferably still from about 350,000 Daltons to about 15,000,000 Daltons.

Depending upon the selection of R, D and R′ the modified polacrylamide polymers each substituents can be charge or neutral, with neutral or cationally charged being preferred. In one preferred embodiment at least 50%, more preferably at least 60%, even more preferably at least 75% of the substituents of the modified polacrylamide polymers have a cationic charge.

Suitable modified polyacrylamide polymers include the Sedipur range of polyacrylamide polymers avalaible from BASF. The most preferred are the Sedipur C types, which are cationic substituted polyacrylamides, such as Sedipur CF 803.

Adjunct Ingredients:

Adjunct ingredients, as used herein, include ingredients that are optional and generally used to enhance the aesthetics or to provide secondary benefits to the presmoistened wipes of the invention.

Perfumes—Perfumes and perfumery ingredients useful in the present compositions and processes comprise a wide variety of natural and synthetic chemical ingredients, including, but not limited to, aldehydes, ketones, esters, and the like. Also included are various natural extracts and essences which can comprise complex mixtures of ingredients, such as orange oil, lemon oil, rose extract, lavender, musk, patchouli, balsamic essence, sandalwood oil, pine oil, cedar, and the like. Finished perfumes can comprise extremely complex mixtures of such ingredients. Finished perfumes typically comprise from about 0.01% to about 2%, by weight, of the detergent compositions herein, and individual perfumery ingredients can comprise from about 0.0001% to about 90% of a finished perfume composition.

When present the perfume comprises from about 0% to about 0.5%, more preferably from about 0.001% to about 0.20%, even more preferably still 0.005% to about 0.15%, by weight of the composition.

Preservatives—The compositions of the present invention preferably comprise one or more preservatives which mainly to protect the substrate from bacterial or fungal contamination. Examples of preservatives include 2-bromo-2-nitro-1,3-propanediol, 2-((hydroxymethyl) (amino)ethanol, propylene glycol, sodium hydroxymethyl amino acetate, formaldehyde and glutaraldehyde, dichloro-s-triazinetrione, trichloro-s-triazinetrione, and quaternary ammonium salts including dioctyl dimethyl ammonium chloride, didecyl dimethyl ammonium chloride, C₁₂, C₁₄ and C₁₆ dimethyl benzyl. Preferred preservatives include 1,2-benzisothiazolin-3-one sold by Avecia Chemicals (Wilmington, Del. 19897), chlorhexidine diacetate sold by Aldrich-Sigma (1001 West Saint Paul Avenue, Milwaukee, Wis. 53233), and KATHON GC II (mixture of 2-methyl-4-isothiazolin-3-one and 5-chloro-2-methyl-4-isothiazolin-3-one) and NEOLONE M (2-methyl-4-isothiazolin-3-one) sold by the Rohm & Haas Company (100 Independence Mall West, Philadelphia, Pa. 19106). When used, preservatives are preferentially present at concentrations of from about 0.0001% to about 0.02%.

Corrosion Inhibitor—The composition of the present invention may optionally contain a corrosion inhibitor. When present the corrosion inhibitor is preferably present from about 0.0001% to about 0.2%, preferably from about 0.0001% to about 0.1%, more preferably from about 0.0005% to about 0.08% by weight of the composition.

While not wishing to be limited by theory, it is believed that the functional role of the corrosion inhibitor is to form ‘in use’ a protective coating layer on any metal components of an implement in which the compositions of the invention are being stored and applied to a hard surface through. Furthermore, the corrosion inhibitor has the additional benefit of providing corrosion inhibition to the surfaces to which it is applied. Such surfaces would include ranges, refrigerators, and any other surface which is wetted in delivering the solution to the hard surface, such as the floor.

It is preferred that the corrosion inhibitor, when present in the composition is selected from the group consisting of alkali metal or alkaline earth salts of silicate (such as layered sodium silicate e.g. δ—Na₂Si₂O₅, known as NaSKS-6 (trade name), available from Hoechst AG), alkali metal or alkaline earth salts of metasilicate, polyvalent ion salts of fatty acids, alkyl phosphates, paraffin, benzotriazole, inorganic salts of Bi²⁺, organosilicates, inorganic salts of Zn²⁺, and mixtures thereof. It is also preferable to minimize the amount of chloride ions present in the composition as higher levels of chloride ions are believed to increase corrosion.

Suitable corrosion inhibitors can also be found in U.S. Pat. No. 4,199,483; U.S. Pat. No. 4,992,195; U.S. Pat. No. 4,098,720; U.S. Pat. No. 5,736,495; U.S. Pat. No. 3,981,780; U.S. Pat. No. 4,292,190; all of which are hereby incorporated by reference.

Other Optional Composition Ingredients

The composition, herein, may further comprise other optional ingredients. Suitable ingredients include, but not limited to: detergent builders, dyes, enzymes, leveling agents, chelating agents, thickening agents, stabilizers, antioxidants, etc.

Suitable detergent builders include those derived from phosphorous sources, such as orthophosphates, pyrophosphates, tripolyphosphates, etc., and those derived from non-phosphorous sources, such as nitrilotriacetates; and the like. Suitable enzymes include lipases, proteases, amylases and other enzymes known to be useful for catalysis of soil degradation. Suitable leveling agents include polysaccharide gum, such as guar gum, xanthan gum, etc.

The total level of such optional ingredients is low, preferably less than about 0.1%, more preferably less than about 0.05%, to avoid causing hazing or filming/streaking problems. It is preferred that any water in the composition, such as in premixed or ready to use solutions, is de-ionized, distilled or softened water.

pH Requirements:

The compositions of the present invention have a pH of from about 5 to about 11, more preferably from about 5.5 to about 10, most preferably from about 6 to about 9. pH measurements are conducted by first squeezing out the aqueous composition from the premoistened wipe. It is found that above about pH11, the soil entrainment system is less effective in sequestering particulate soil. Below about pH 5, the acidity of the chemical composition embedded in the substrate competes for anionic sites on the substrate, thereby weakening the ability of the soil entrainment system to anchor.

### BENEFITS & APPLICATIONS

While the benefits of the soil entrainment system have been previously been observed in the context of detergent, unexpected benefits have been found when the entrainment soil system is included in a premoistened wipe. That is, soil removal, and more specifically particulate soil removal, is enhanced when the soil entrainment system is impregnated on a nonwoven substrate.

While not intending to be bound by any theory, it is believed that premoistening the substrate allows the soil entrainment system to better anchor on the nonwoven, thus increasing the soil entrainment system effectiveness. It is believed that such anchoring mechanism is partly due to the interactions between cationic nitrogens of the entrainment system and anionic sites available in the nonwoven substrate. As such, the entrainment system preferably has a high molecular weight, and the nonwoven substrate includes some amount of cellulose-derived fibers. Benefits are observed not only in terms of greater soil pick-up, but also, noticeably more soiled and blackened substrate following the cleaning task. This is important, as floor manufacturers intentionally makeflooring that effectively hides particulate dirt. This is achieved through the use of greyish colours and non-uniform floor patterns. As a result, consumers can have a difficult time judging the performance of a cleaning product by visual examination of the cleaned surface. They often judge the efficacy of a premoistened wipe or other cleaning implement by the appearance of the dirty wipe.

Directly attaching the soil entrainment system onto substrate via the creation of a premoistened wipe offers additional benefits. First, it limits the amount of the entrainment system that remains on the surface to be cleaned. The soil entrainment systems of this invention are all polymers with cationic charge and these will leave visible residues and/or adhere to the floor if not removed by the cleaning implement during the cleaning process. Second, it allows for use of enhanced levels of the soil entrainment system. This is often not possible when the soil entrainment system is directly placed on the surface to be cleaned rather than on the substrate, because of enhanced floor stickiness. For premoistened wipes, the benefits of higher levels of soil entrainment system can also be realized by simply increasing the solution load factor. Finally, efficiency is improved leading to lower cost spent on these expensive polymeric systems.

‘Glass’ Cleaning Uses:

One important application of the cleaning system disclosed herein is in the cleaning of windows, mirrors, glass tables, and other surfaces such as home shelves and blinds, which tend to be lightly soiled and where the soil content is predominantly particulate matter (as opposed to, fro example, greasy soils). In this instance, the soil entrainment system is critical to reduce, more preferably eliminate, haze caused by the redistribution of soils that can occur as the wiping implement sweeps across the surface to be cleaned. While any nonwoven substrate can be used, premoistened glass-and-surface wipes will preferably include a substrate that comprises no more than about 80% of synthetic fibers. The preference for inclusion of some cellulose-based fibers lies in the relative simplicity of the task, the high particulate soil and the desire to maximize adsorption of the entrainment system on the substrate so as to limit filming and streaking issues due to the soil entrainment system. In most cases, the substrate will comprise from about 20% to about 80% synthetic fibers, thus providing good glide across the glass surface and good mileage. In a preferred embodiment the wipe will have a basis weight of from about 20 gm⁻² to about 120 gm⁻², preferably from about 25 gm⁻² to about 100 gm⁻², more preferably from about 30 gm⁻² to about 80 gm⁻² and have a liquid load factor of from about 1:1 to about 2.5:1, more preferably 1.1:1 to about 2.2:1, most preferably 1.2:1 to 2.0:1. The low load factor ensures the delivery of the necessary amount of fluid for cleaning without excessive streaking or filming. In a preferred embodiment, the soil entrainment system is present at levels of from about 0.001% to about 0.20%, preferably from about 0.005% to about 0.15%, more preferably from about 0.01% to about 0.10%, by weight of the detergent composition. Preferred surfactants for use in glass cleaning applications include sulfobetaines, betaines and alkyl polyglucosides at concentrations from about 0.005% to about 0.20%, preferably from about 0.01% to about 0.25%, more preferably from about 0.02% to about 0.10%. In addition, cleaning solvents including C3-C6 glycol ethers are preferably included at levels of from about 0.25% to about 5%, and volatile solvents such as ethanol, isopropanol and methoxypropanol are preferably present at levels of from 0.25% to about 5%. Other highly desirable adjuncts include preservatives and dyes.

‘Counter’ Cleaning Uses:

Premoistened wipes are well suited for the cleaning of many home surfaces including kitchen countertops, bath sinks, or wood tables, walls, and the like. For purposes of this invention, these various applications are grouped under the heading ‘counter’ cleaning. In this context the substrate preferably comprises at least about 20% synthetic fibers, more preferably from about 30% to about 100% synthetic fibers. Counter cleaning wipes can consist of a single substrate layer or of a laminate of two or more layers. When two or more layers are included, it is preferred that at least one of the outer layers be smooth while the other can have either a smooth structure or a macroscopically visible 3-dimensional structure. Such 3-dimensional structure can be created via any means known in the art, such as inclusion of fibers of various denier or inclusion of a scrim The premoistened wipe for counters has similar physical properties as described for glass cleaning applications, but includes a higher load factor. The load factor is preferably from about 2:1 to about 8:1, more preferably from about 2.5:1 to about 6:1, most preferably from about 3:1 to about 5:1. The level and type of entrainment system and other adjuncts is similar to that for the glass cleaning application.

‘Floor’ Cleaning Uses:

In another embodiment, the premoistened wipe of the present invention is used for cleaning floor surfaces. Such surfaces include vinyl, linoleum, ceramic, marble, slate, wood and the like. Particulate soil is very common on floors and soil levels are generally higher than what is observed on glass. Additionally, floor sizes can vary significantly, depending on home size and amount of carpeted area. As such, several types of wipes can be used. For small areas such as bathroom floors, the premoistened wipe can consist of a substrate monolayer comprising at least 20% synthetic fibers and a basis weight of from about 50 gm⁻² to about 200 gm⁻², preferably from about 70 gm⁻² to about 170 gm⁻², more preferably from about 90 gm⁻² to about 150 gm⁻². The substrate is then loaded with a composition comprising the soil entrainment system at a load factor of from about 3:1 to about 12:1, more preferably from about 3.5:1 to about 10:1, most preferably from about 4:1 to about 8:1.

For cleaning larger floor areas, including kitchen surfaces, living room surfaces, bedroom floors and combinations thereof, the premoistened wipe is preferably a laminate. A suitable example of a premoistened wipe laminate is SWIFFER WET® sold by The Procter & Gamble Company. Laminates will preferably comprise a floor sheet layer with a basis weight of from about 15 gm⁻² to about 80 gm⁻², preferably from about 20 gm⁻² to about 75 gm⁻², more preferably from about 25 gm⁻² to about 70 gm⁻². The floor sheet preferably comprises at least about 20% synthetic fibers, preferably about 30%, more preferably at least about 40%. A second layer comprises mainly pulp or paper-derived fibers and acts as a fluid reservoir and dispensing unit to the floor sheet. Preferably, the fluid reservoir has a basis weight of from about 100 gm⁻² to about 300 gm⁻², more preferably from about 100 gm⁻² to about 200 gm⁻². Other layers may optionally be included to help fluid dosing purposes or, as an attachment layer. In general, the wipe area, caliper, fiber density and basis weights of the various layers in the wipe will define the load factors available for the cleaning composition. Preferably, the cleaning composition and entrainment system are embedded into the overall wipe structure (i.e., the combination of all layers) at a load factor of from 4:1 to about 15:1, more preferably from about 5:1 to about 12.5:1, most preferably from about 6:1 to about 10:1.

### TEST METHODS

General:

Two test methods employed herein to illustrate the benefits of the compositions of the present invention The first test method applies to premoistened ‘counter’ wipes and ‘floor’ wipes and illustrates the cleaning ability of the inventive premoistened wipes on a ceramic surface. The second test method illustrates the cleaning ability of the premoistened wipes of the invention in the context of a ‘glass’ wipe. The chemical compositions and substrates used for the two test methods are listed below.

Chemical Compositions:

| | % w/w | | | | | | |
|---|---|---|---|---|---|---|---|
| | A | B | C | D | E | F | G |
| C10 Alkyl Polyglucoside* | 0.06 | 0.06 | | | 0.05 | 0.05 | 0.05 |
| Cocamidopropyl sulfobetaine | | | 0.05 | 0.05 | | | |
| Ethoxylated castor oil | | | | | 0.01 | 0.01 | 0.01 |
| Propylene glycol n-butyl ethe | 1 | 1 | 1 | 1 | 1 | 1 | 1 |
| Polydimethyl siloxane** | 0.00375 | 0.00375 | 0.00375 | 0.00375 | 0.00375 | 0.00375 | 0.00375 |
| Lupasol SK*** | 0.02 | | 0.02 | | 0.02 | 0.02 | |
| 1,2-Benzisothiazolin-3-one | 0.015 | 0.015 | 0.015 | 0.015 | 0.015 | 0.015 | 0.015 |
| Perfume | 0.05 | 0.05 | 0.05 | 0.05 | 0.05 | 0.05 | 0.05 |
| PHMB**** | | | | | 0.2 | | 0.2 |

*Plantaren 2000 N UP (Cognis)
**Dow Corning AF suds suppressor (Dow Corning)
***Soil entrainment polymer (BASF)
****Poly(HexaMethyleneBiguanide) from Vantocil IB (Avecia Chemicals)

All of the above chemical compositions have pH from about 7 to about 9. As delivered from premoistened wipes, the pH of the cleaning compositions above, expressed from premoistened wipes, ranged from about 7 to about 8.

Substrates:

| | |
|---|---|
| L | 100% Polyester (Synthetic), 60 gsm substrate with |
| | lotion at 4.0x load factor |
| M | 60/40 PP/Lyocell, 60 gsm substrate with lotion at |
| | 4.0x load factor |
| N | 100% Rayon, 60 gsm substrate with lotion at 4.0x |
| | load factor |
| P | 70/30 Rayon/PET (Mixed fibers), 105 gsm substrate |
| | with lotion at 4.0x load factor |
| Q | Trilaminate (Swiffer Wet current) pad with 7.0x |
| | load factor |
| R | Spray solution and sponge |
| S | Spray solution and Clorox Ready Mop Pad |

Grading:

Grading was performed on a 0-4 scale according to the following scale:

0: the tile appears completely clean; no visible residues of any kind are seen with the naked eye

1: the tile is generally clean; low levels of residue, either streaks or film, are visible but faint

2: the tile is not clean; residues, either streaks or film, are clearly visible

3: the tile is very dirty; residues, either streaks or film, substantially coat the tile are very visible

4: the tile is completely coated with highly visible residue.

‘Counter’ and ‘Floor’ Wipe Test Methodology

Parma Ceramicraft glazed black ceramic tiles (size 30.5 cm*30.5 cm) manufactured by Marazzi (France)_are cleaned with a solution of isopropanol and water and then wiped dry. The tile type was chosen so as to make visualization of the end result easier. It makes the testing more objective than simply comparing the level of soil locked away by the different wipes (more soiling can qualitatively be seen for wipes comprising the entrainment system). Wipes are prepared by adding cleaning composition directly onto dry substrate and spreading the cleaning compositions to completely cover the substrate and ensure uniformity of distribution. The load factor employed is dependent on the choice of substrate and product application. For the monolayer wipes (for cleaning counters and the like), a load factor of 4:1 is used; for the floor cleaning laminate (Swiffer Wet pad type), a load factor of 7:1 is used. The premoistened wipes are then stored at ambient conditions for a minimum of 12 hours prior to testing. All monolayer wipes tested had dimensions of about 16 cm*9 cm. The dimensions of the laminate premoistened wipe Q tested is 8.7 cm*10.5 cm (one Swiffer Wet pad cut in three equal sized piece along the width of the wipe).

The premoistened wipes comprising the soil entrainment system were tested versus similar premoistened wipes that lacked the soil entrainment system. Thus, the difference in average grade provides a measure of the benefits associated with the soil entrainment system.

So as to yet better illustrate the inventiveness of the compositions, premoistened wipes are also tested versus spray-and-wipe systems. When used, the sponges disclosed herein are common commercial materials (SPONTEX) cut to dimensions 8cm*4 cm*3cm.8cm They weigh 4.5±0.5 grams dry and are loaded with about 8-10 grams of water prior to use. Clorox Ready Mope pads, also used in the testing, were cut to dimensions 9 cm*11 cm (Clorox Ready Mope pads are cut into thirds along the pad width) and used as is.

The test soil is spread uniformly on the second tile to be cleaned by each premoistened wipe composition. The soil consists of a mixture of oil and polymerized grease, sugar and particulate soil. About half the soil is particulate matter. In a glass beaker comprising a magnetic stirrer, 120 grams isopropanol and 50 grams de-ionized water were blended together. Four grams of soil (about 50% particulate matter) were then added under continued agitation. All soil transfers were conducted while the soil solution was under agitation. For soiling tiles, 1 gram of soil solution is placed in an aluminum foil boat. A primed roller is then rolled over the aluminum foil until the soil solution is transferred to the roller. The soiled roller is then used to transfer soil to the relevant test tiles.

For each premoistened wipe (or spray-and-wipe system), the testing procedure herein described is performed twice to ensure validity of the results. Each test consists of wiping three tiles in succession with the product of interest. The wipe pattern used consists of eight overlapping strokes (four up the tile, four down the tile) from right to left followed by eight overlapping strokes (four up the tile, four down the tile) from left to right. In the case of spray-and-wipe systems, the aqueous compositions tested (sponges or disposable absorbent pads) are carefully pipetted along one of the edges (2-3 cm away) such that the line of aqueous product placed on the tile is at a 90° angle to the main up-and-down wiping motion. This provides reassurance that the implements evenly distribute cleaning composition across the tiles. The amount of liquid placed is adjusted such that the average amount of liquid lost to the tiles after wiping was comparable to that lost when testing is conducted with premoistened wipes. With equal amounts of fluid left on tile using identical solutions, direct comparisons of the benefits of the entrainment system for premoistened wipes versus spray-and-wipe systems can be made.

For each product tested (either premoistened wipe or spray-and-wipe product), three tiles are wiped in succession. In each case, the first tile wiped is initially clean; the second tile wiped is pre-soiled and the third tile wiped is initially clean. Results from the first tile cleaning provide an indication of residue (Filming/Streaking) left on tile due to the cleaning composition; results from the second cleaning testify to the raw cleaning ability of the premoistened wipe, and results from the third tile show the amount of redeposited soil. All compositions are tested blind (i.e., the number of tiles employed was a multiple of 3, and expert graders are used to grade each the tiles). The overall score of a product is computed as the sum of the scores for each three tile set. Lower overall scores suggest improved performance.

‘Glass’ Wipe Testing Methodology:

Four windows (66 cm width×81 cm height) are mounted about 100 cm from the ground. Each window is cleaned thoroughly (on both sides of the window pane) by first applying Cinch® glass cleaner solution and wiping with Bounty® paper towel. The dry window is then cleaned with an isopropyl alcohol/water mixture and wiped dry again with Bounty® paper towel. This step removed unwanted surfactant from the first cleaning process. The window is then saturated with distilled water (once again, both sides) and cleaned with a squeegee to dryness. The procedure is repeated until it can be ascertained that all windows are soil-, streak-, and film-free.

The cleaned windows are then soiled with particulate soil from a well mixed spray bottle comprising 0.50 grams total particulate in 500 grams distilled water. Approximately 5 milliliters of soil solution are sprayed over each of the test windows. The soil solution is then allowed to dry (30 minutes).

Each product is tested twice to ensure reproducibility of results. Premoistened wipe M with dimensions 16 cm*27 cm is uniformly loaded at a 1.5:1 load factor with cleaning compositions A, B, C and D disclosed above and kept for at least 12 hours before use. Each of the test wipes is folded in half (new dimensions are 16 cm*13.5 cm) for use purposes. Window wiping is accomplished by wiping up-and-down along the length of the window in 10 up-and-down overlapping strokes moving from the the left of the window to the right side (five strokes up and five strokes down), followed by 12 strokes from side to side moving from the upper part of the window to the lower part of the window. The wipe is then folded back on itself exposing a clean (unused) portion of the wipe. The freshly exposed clean portion of the premoistened wipe is then used to re-wipe the once-wiped soiled window in identical fashion as just described (i.e., 10 up-and-down strokes followed by 12 side-to-side strokes).

### EXAMPLES

The following non-limiting examples illustrate the innovative compositions of the present invention.

Results For Monolayer Premoistened Wipes (‘Counter’ Wipes):

| | Tile | | | | | |
|---|---|---|---|---|---|---|
| Compo- | 1: | Tile 2: | Tile 3 | Grade | Grade | Avg. Soln. |
| sition | F/S | Cleaning | Redep | sum | Diff (B − A) | On Tile |
| AL | 0.4 | 2.0 | 1.3 | 3.6 | | 1.5 |
| BL | 0.3 | 2.5 | 1.3 | 4.0 | 0.4 | 1.2 |
| AM | 0.3 | 2.7 | 1.5 | 4.5 | | 1.2 |
| BM | 0.5 | 2.7 | 1.6 | 4.8 | 0.3 | 1.1 |
| AN | 0.4 | 1.6 | 1.2 | 3.2 | | 1.0 |
| BN | 0.7 | 2.4 | 1.8 | 4.8 | 1.7 | 1.1 |
| AP | 0.2 | 1.9 | 1.5 | 3.5 | | 1.2 |
| BP | 0.6 | 2.1 | 1.8 | 4.5 | 1.0 | 0.9 |

For each substrate L, M, N and P, the composition comprising the soil entrainment system shows improved overall end result (sum column) versus comparable compositions lacking the soil entrainment system. Additionally, the magnitude of the benefit provided by the premoistened wipes is largest for substrates N and P, which are high in rayon content. The larger benefit observed for these substrates is ascribed to increased affinity of the cationic soil entrainment system for the cellulosic fibers.

Results for Floor Cleaning Premoistened Wipes:

(i) Tests With Alkyl Polyglucoside Surfactant

| | Tile | | | | | |
|---|---|---|---|---|---|---|
| Compo- | 1: | Tile 2: | Tile 3 | Grade | Grade | Avg. Soln. |
| sition | F/S | Cleaning | Redep | sum | Diff (B − A) | On Tile |
| AQ | 0.9 | 1.8 | 0.6 | 3.3 | | 3.6 |
| BQ | 1.6 | 2.2 | 1.3 | 5.2 | 1.9 | 3.1 |
| AR | 0.0 | 2.5 | 1.5 | 4.1 | | 3.8 |
| BR | 0.3 | 2.6 | 1.4 | 4.3 | 0.2 | 3.7 |
| AS | 0.3 | 2.7 | 1.5 | 4.5 | | 4.3 |
| BS | 0.3 | 2.8 | 1.8 | 4.9 | 0.4 | 4.2 |

Incorporation of the soil entrainment system in the composition impregnated on substrate Q provides a large end result benefit versus the comparable premoistened wipe composition lacking the entrainment system. Not only is the magnitude of the soil entrainment system benefit for the premoistened wipe larger than it is for the spray-and-wipe systems, the data suggest that the actual end result for the premoistened wipe also the best (compare the sum column for compositions AQ vs. AR and AT). The strength of composition AQ vs. all others is particularly large for cleaning and redeposition.

(ii) Tests with Sulfobetaine Surfactant

| | Tile | | | | | |
|---|---|---|---|---|---|---|
| Compo- | 1: | Tile 2: | Tile 3 | Grade | Grade | Avg. Soln. |
| sition | F/S | Cleaning | Redep | sum | Diff (B − A) | On Tile |
| CQ | 0.0 | 1.7 | 1.0 | 2.8 | | 3.9 |
| DQ | 0.8 | 2.3 | 1.6 | 4.6 | 1.8 | 3.6 |
| CR | 0.0 | 2.4 | 1.2 | 3.6 | | 3.7 |
| DR | 0.2 | 2.7 | 1.6 | 4.5 | 0.9 | 3.6 |
| CS | 0.2 | 2.6 | 1.8 | 4.6 | | 4.3 |
| DS | 0.3 | 3.0 | 2.1 | 5.3 | 0.7 | 4.3 |

Changing the surfactant system from C10 APG to Cocamidopropyl sulfobetaine has little impact on the observed trends. That is, the largest benefits associated with the incorporation of the soil entrainment system are observed in the context of the premoistened wipe. The best overall end result is achieved by the premoistened wipe comprising the soil entrainment system (compare the sum column for each of the compositions).

Results for Compositions Comprising Polymeric Biguanides:

(i) Counter and Floor Cleaning Wipes

| | Tile | | Tile 3 | | Grade | Grade | Avg. |
|---|---|---|---|---|---|---|---|
| Compo- | 1: | Tile 2: | Re- | Grade | Diff | Diff | Soln. |
| sition | F/S | Cleaning | dep | sum | (F − E) | (G − E) | On Tile |
| EM | 0.4 | 3.2 | 2.3 | 5.8 | | | 1.2 |
| FM | 0.3 | 3.5 | 2.1 | 5.9 | 0.1 | | 1.0 |
| GM | 1.2 | 3.5 | 2.4 | 7.0 | | 1.2 | 0.9 |
| EQ | 0.6 | 1.5 | 0.8 | 2.9 | | | 3.6 |
| FQ | 0.8 | 2.2 | 1.0 | 4.0 | 1.1 | | 3.6 |
| GQ | 1.8 | 2.5 | 1.9 | 6.2 | | 2.2 | 3.1 |

Compositions with substrate Q provide much larger end result benefits vs. comparable compositions with substrate M. These benefits are ascribed to increased amount of solution wiped on the tile (due to higher load factor), which helps overall cleaning and magnifies the benefits of the soil entrainment system. Nevertheless, the performance trend for wipes M and Q is the same: the best performance is achieved by the combination of soil entrainment system and polymeric biguanide, and the worst performance takes place in the absence of the soil entrainment system. In addition to providing improvement in end result performance, the polymeric biguanide is also an active disinfectant. For example, wipes EQ and GQ have been tested using US EPA protocol and found to be fully cidal vs. Staphylococcus aureus and Salmonella choleraesuis. The incorporation of the soil entrainment system into compositions comprising polymeric biguanides is therefore highly beneficial, delivering excellent antimicrobial properties and excellent end result cleaning in a single execution.

(ii) Spray-and-Wipe Systems

| | | | | | Grade | Grade | Avg. |
|---|---|---|---|---|---|---|---|
| | Tile 1: | Tile 2: | Tile 3 | Grade | Diff | Diff | Soln. |
| | F/S | Cleaning | Redep | sum | (F − E) | (G − E) | On Tile |
| ER | 0.1 | 2.0 | 1.0 | 3.1 | | | 3.5 |
| FR | 0.7 | 3.0 | 1.5 | 5.2 | 2.1 | | 3.4 |
| GR | 1.0 | 3.4 | 1.8 | 6.3 | | 3.2 | 3.7 |
| ES | 0.2 | 1.0 | 0.5 | 1.7 | | | 4.2 |
| FS | 0.3 | 2.1 | 1.5 | 3.8 | 2.1 | | 4.5 |
| GS | 0.5 | 1.4 | 1.3 | 3.2 | | 1.5 | 4.6 |

As in the case of the premoistened wipes, spray-and-wipe systems also show surprising synergy between the soil entrainment system and polymeric biguanide. The benefits of having both raw materials in the overall composition are observed for conventional cleaning systems (i.e., sponges) as well as absorbent disposable wipes (Clorox Ready Mop® pad).

Glass Cleaning Composition Test Results

| | | | Avg. | Diff | |
|---|---|---|---|---|---|
| Composition | Test 1 | Test 2 | grade | (B − A)) | Diff (D − C) |
| AM | 1.4 | 1.3 | 1.4 | | |
| BM | 2.7 | 2.9 | 2.8 | 1.4 | |
| CM | 1.6 | 1.8 | 1.7 | | |
| DM | 2.8 | 3.0 | 2.9 | | 1.2 |

Once again, the premoistened wipes comprising the soil entrainment system once again show marked benefits versus equivalent products that do not comprise without the soil entrainment system.

While particular embodiments of the subject invention have been described, it will be apparent to those skilled in the art that various changes and modifications of the subject invention can be made without departing from the spirit and scope of the invention. In addition, while the present invention has been described in connection with certain specific embodiments thereof, it is to be understood that this is by way of limitation and the scope of the invention is defined by the appended claims which should be construed as broadly as the prior art will permit.

What is claimed is:

1. A premoistened wipe comprising:
a) at least one layer of a nonwoven substrate, wherein said layer is made of a material selected from the group consisting of synthetic fibers, natural fibers and mixtures thereof;
b) a cleaning composition impregnated in said nonwoven substrate and loaded at weight factor of from about 1:1 to about 15: 1, comprising an effective amount of a soil entrainment system, said soil entrainment system is selected from the group consisting of
1) one or more modified polyamine compounds, said modified polyamine compounds are selected from:
i) (PA)_{w}(T)ₓ;
ii) (PA)_{w}(L)_{z};
iii) [(PA)_{w}(T)ₓ]_{y}[L]_{z}; and
iv) mixtures thereof;
wherein PA is a grafted or non-grafted, modified or unmodified polyamine backbone unit, T is an amide-forming polycarboxylic acid crosslinking unit, and L is a non-amide forming crosslinking unit; provided that for compounds of type (i) and (iii) the indices w and x have values such that the ratio of w to x is from 0.8: 1 to 1.5: 1; for compounds of type (ii) the indices w and z have values such that said modified polyamine compound comprises from about 0.05 to about 2 parts by weight of said L unit; for compounds of type (iii) the indices y and z have values such that said modified polyamine compound comprises from about 0.05 to about 2 parts by weight of said L unit; and
2) one or more modified polyacrylamide compounds of the formula:
—[CR₂—CR₂]ₙ—
wherein each R unit is independently selected from the group consisting of hydrogen, C₁-C₁₂ alkyl, C₆-C₁₂ aryl, CON(R′)₂, and D; wherein each D unit is independently selected from the group consisting of CO₂N(R′)ₘ, CON(R′)CH₂CON(R′)₂, OCON(R′)₂, and CO₂(CH₂)_{q}N(R′)ₘ, wherein each R′ is selected from the group consisting of hydrogen, C₁-C₁₂ alkyl, or both R′ units can be taken together to form a ring comprising 4-6 carbon atoms; q is an integer from 0 to 5; m is either 2 or 3 and n is a numbers selected such that said modified polyacrylamide compounds have an average molecular weight of from about 100,000 Daltons to about 10,000,000 Daltons; and
3) mixtures thereof; and
c) the balance adjunct ingredients;
wherein said cleaning composition has a pH as expressed from said premoistened wipe of from about 5 to about 11.

2. The premoistened wipe of claim 1 wherein the substrate comprises from about 20% synthetic fibers to about 80% synthetic fibers.

3. The premoistened wipe of claim 1 wherein the cleaning composition comprises at least one surfactant selected from the group consisting of nonionic, zwitterionic and amphoteric surfactants.

4. The premoistened wipe of claim 3 wherein said surfactant is a nonionic or zwitterionic surfactant selected from the group consisting of alkyl polyglucosides, betaines and sulfobetaines.

5. The premoistened wipe of claim 1 wherein the cleaning composition comprises from about 0.005% to about 0.5% by weight of said soil entrainment system.

6. The premoistened wipe of claim 1 wherein said substrate has a basis weight of from about 20 gm⁻² to about 120 gm⁻² and wherein said cleaning composition is loaded onto said substrate at a load factor of from about 1:1 to about 2.5:1.

7. The premoistened wipe of claim 1 wherein said substrate has a basis weight of from about 50 gm⁻² to about 200 gm⁻².

8. The premoistened wipe of claim 1 wherein the substrate is a laminate comprising:
i) a floor sheet layer having a basis weight of from about 15 gm⁻² to about 80 gm⁻²and
ii) at least one reservoir layer having a basis weight of from about 100 gm⁻² to about 300 gm⁻².

9. The premoistened wipe of claim 7 wherein said laminate is loaded with the cleaning composition at a load factor of from about 4:1 to about 15:1.

10. The premoistened wipe of claim 1 wherein said cleaning composition further comprises one or more antimicrobial agents.

11. The premoistened wipe of claim 10 wherein said antimicrobial agent is a polymeric biguanide.

12. The premoistened wipe of claim 11 wherein said polymeric biguanide is a poly(hexamethylene biguanide) or a salt thereof.

13. An aqueous hard surface cleaning composition comprising:
a) an effective amount of a soil entrainment system, said soil entrainment system is selected from the group consisting of
1) one or more modified polyamine compounds, said modified polyamine compounds are selected from:
i) (PA)_{w}(T)ₓ;
ii) (PA)_{w}(L)_{z};
iii) [(PA)_{w}(T)ₓ]_{y}[L]_{z}; and
iv) mixtures thereof;
wherein PA is a grafted or non-grafted, modified or unmodified polyamine backbone unit, T is an amide-forming polycarboxylic acid crosslinking unit, and L is a non-amide forming crosslinking unit; provided that for compounds of type (i) and (iii) the indices w and x have values such that the ratio of w to x is from 0.8:1 to 1.5:1; for compounds of type (ii) the indices w and z have values such that said modified polyamine compound comprises from about 0.05 to about 2 parts by weight of said L unit; for compounds of type (iii) the indices y and z have values such that said modified polyamine compound comprises from about 0.05 to about 2 parts by weight of said L unit; and
2) one or more modified polyacrylamide compounds of the formula:
—[CR₂—CR₂]ₙ—.
wherein each R unit is independently selected from the group consisting of hydrogen, C₁-C₁₂ alkyl, C₆-C₁₂ aryl, CON(R′)₂, and D; wherein each D unit is independently selected from the group consisting of CO₂N(R′)ₘ, CON(R′)CH₂CON(R′)₂, OCON(R′)₂, and CO₂(CH₂)_{q}N(R′)ₘ, wherein each R′ is selected from the group consisting of hydrogen, C₁-C₁₂ alkyl, or both R′ units can be taken together to form a ring comprising 4-6 carbon atoms; q is an integer from 0 to 5; m is either 2 or 3 and n is a numbers selected such that said modified polyacrylamide compounds have an average molecular weight of from about 100,000 Daltons to about 10,000,000 Daltons; and
3) mixtures thereof; and
b) at least one antimicrobial agent wherein said antimicrobial agent is a polymeric biguanide; and
c) the balance adjunct ingredients;
wherein said cleaning composition has a pH as expressed from said premoistened wipe of from about 5 to about 11.

14. The cleaning composition of claim 13 wherein said polymeric biguanides is a poly(hexamethylene biguanide).or salt thereof.

15. A method of cleaning a hard surface comprising the step of contacting said hard surface with the premoistened wipe of claim 1.

16. A method of cleaning a hard surface comprising the step of contacting said hard surface with the aqueous composition of claim 13.

17. The method of claim 16 further comprising the step of wiping said hard surface with a disposable absorbent pad.
